(19) United States
(12) Patent Application Publication
(10) Pub. No.: US 2026/0076677 A1
(43) Pub. Date: Mar. 19, 2026

(54) ENDOSCOPIC METHODS AND SYSTEMS FOR GASTROINTESTINAL RESHAPING AND RESURFACING

(71) Applicant: ENDOLASTIC, INC., Raleigh, NC (US)

(72) Inventors: Frederick Haller, Winston-Salem, NC (US); Paul Robertson, Raleigh, NC (US)

(21) Appl. No.: 19/331,122

(22) Filed: Sep. 17, 2025

Related U.S. Application Data
(60) Provisional application No. 63/696,001, filed on Sep. 18, 2024.

(51) Int. Cl. A61B 17/12; A61B 1/00; A61B 1/273; A61B 17/00; A61F 5/00

(52) U.S. Cl. CPC A61B 17/12013; A61B 1/00094; A61B 1/273; A61F 5/005; A61F 5/0089; A61B 2017/00544; A61B 2017/00561; A61B 2017/00827; A61B 2017/00862; A61B 2017/12018

(57) ABSTRACT

Non-resective endoscopic systems and methods for reshaping gastrointestinal organs to treat gastroesophageal reflux disease (GERD), obesity, Type 2 Diabetes Mellitus, and perforations. The non-resective endoscopic systems and methods provide improved tissue capture capacity to create pressure necrosis to target tissue areas. The non-resective endoscopic systems and methods include banding target tissue areas utilizing a ligation band. The ligation band creates a uniform distribution of pressure to improve tissue retention and pressure necrosis for eliciting a healing response.

### CROSS-REFERENCE TO RELATED APPLICATION

This application claims the benefit of and priority to U.S. Provisional Patent Application No. 63/696,001 , filed on Sep. 18, 2024, the entire content of which is herein incorporated by reference in its entirety.

### BACKGROUND

The gastroesophageal junction (GEJ) hinders the flow of the gastric contents into the esophagus to provide a barrier against gastroesophageal reflux. The GEJ provides a high-pressure zone created by a smooth muscle of the lower esophageal sphincter (LES), the skeletal muscle of the crural diaphragm, in combination with clasp and sling fibers in the gastric cardia. GEJ issues can be caused by Gastroesophageal Reflux Disease (GERD). GERD relates to the malfunctioning of the lower esophageal sphincter (LES) that results in stomach contents (e.g., stomach acid) leaking into the esophagus. Once the stomach acid contacts the esophagus lining, a burning sensation results (e.g., heartburn). GERD impairs physical and psychological quality of life and affects approximately one billion individuals.

Current treatment of GERD includes a Laparoscopic Nissen Fundoplication (“Nissen”) and a Stretta procedure. The surgical Nissen treatment is invasive, expensive, and can have postoperative complications. The Nissen treatment is an invasive procedure that involves a partial wrap of the proximal stomach around the esophagus. Commonly occurring side effects of the Nissen procedure can significantly impair the ventilation of swallowed air. Additionally, there is non-negligible morbidity like dysphagia, inability to belch or vomit, and increased bloating and flatulence called gas bloat syndrome. The endoscopic Stretta procedure involves the use of radiofrequency ablation (RFA) applied to the GEJ. While the Stretta procedure can treat GERD, it has high cost and training that provides a barrier to the modality.

Other FDA cleared endoscopic treatments include transoral incisionless fundoplication (TIF) and gastroesophageal (GE) junction plication. Although these treatments are promising, the treatments lack long-term efficacy data and are expensive and cumbersome because costly proprietary capital equipment is required.

Anti-Reflux Mucosal Resection (ARMS) is an experimental GERD treatment technique. This approach utilizes existing, relatively inexpensive tools that require no additional endoscopic training. Mucosal tissue is resected with an endoscopic barrel and subsequently resected using a snare in order to achieve a tightening of the lower esophageal sphincter by submucosal fibrosis induced after extensive mucosectomy of the GEJ. A successful ARMS procedure has been demonstrated in many live scenarios with positive acceptance and takes about an hour to perform as pre-marking the treatment site and saline injection are both part of the procedural protocol.

Recently, there have been a few promising endoscopic treatments developed with the goal of reducing the diameter of the lower esophageal sphincter (LES), mimicking the surgical approach, without the associated invasiveness and side effects. Unfortunately, these techniques currently suffer from lack of efficacy, high cost, extensive training requirements and the need for further development.

Another way of treating GERD is ligation, particularly endoscopic ligation. Endoscopic band ligation is an endoscopic procedure involving the application of an apparatus (e.g., thread, wire, band) to constrict tissue in a body to prevent blood flow. Typically, using an endoscope, physicians use suction to pull varices into a chamber at the end of the endoscope and wrap them with an elastic band. This prevents the veins from bleeding. Failure to properly treat these veins may cause them to rupture and result in serious bleeding. Ligation can treat varices, polyps, hemorrhoids, and other lesions. Endoscopic band ligation uses elastic bands to treat enlarged veins in a person's esophagus. Ligation bands need sufficient elasticity and gripping force to avoid band slippage during and after endoscopic procedures.

Endoscopic treatments offer the opportunity to provide a more cost effective, accessible, minimally invasive intervention that can function as a viable alternative to lifelong proton pump inhibitors (PPI) use and anti-reflux surgery (ARS). To date, however, no endoscopic procedure has been widely accepted as a standard treatment for GERD due to insufficient symptom control or the requirement of costly, cumbersome devices linked to time consuming technically challenging procedures. Therefore, there is a need for a ligation band that improves endoscopic treatments of GERD.

Anti Reflux Band Mucosectomy (ARBM) is a treatment technique for GERD that uses only band ligation. This method uses natural pressure necrosis applied to the tissue of the lower esophageal sphincter. This band-only technique eliminates the cautery resection procedural step of the endoscopic mucosal resection (EMR). This cautery resection step is generally associated with bleeding, stricturing, and rare but dangerous perforations. Current ligation bands for variceal ligation have been repurposed for this ARBM GERD procedure. This ligation GERD procedure was effective; however, current ligation bands need to be customized for the GERD procedure. The ligation bands need to be designed for the purpose of capturing muscle fibers within the band space and retaining those fibers in order to have a more durable outcome.

Current ligation bands are designed only to retain and restrict mucosal and submucosal tissue layers. These ligation bands are not capable of retaining the underlying muscle tissue layers. With the possibility of the ARBM procedure for GERD, there is a need for a novel ligation band that improves ligation based endoscopic treatments for GERD. This proposed ligation band will need to have the ability to retain mucosal, submucosal, and more importantly, muscle layers inside of the band for maximum therapeutic effect and durability.

In addition to GERD, endoscopic treatments are also used to treat obesity. Current endoscopic treatment for obesity includes using an intragastric balloon (IGB) and endoscopic sleeve gastroplasty (ESG). The balloons have reasonable clinical efficacy when combined with a multi-disciplined support program. However, the ballons can cause gastric pain, GERD, and blockage. ESG procedures require extensive training and time to successfully perform. Alternatively, ligation bands may be used. Ligation bands have significantly lower risk, cost, and training requirements. Current ligation bands used for endoscopic procedures fail to generate enough retention capability to capture the muscularis propria layer. The muscularis propria layer is necessary to create an effective fibrotic effect for reshaping the greater curve of the stomach.

Conventional endoscopic treatments result in insufficient tissue capture and resection methods. These endoscopic treatments may not provide long-term solutions for gastrointestinal conditions including GERD and obesity. This is due in part to typical endoscopic barrels capturing less than 750 mm³ of tissue. These endoscopic systems and devices fail to hold the muscularis propria tissue. There is a need for an improved gastrointestinal reshaping endoscopic system and method that captures and retains large tissue volumes effectively and safely, without the need for tissue resection.

In addition to GERD and obesity, iatrogenic perforations and Type 2 Diabetes Mellitus conditions are treatable with endoscopic systems and methods. Iatrogenic perforation occurs when the integrity of the gastrointestinal tract wall is compromised during endoscopic procedures. The perforation can lead to peritonitis, sepsis, and other life-threating complications. Traditional surgical repair, while effective, involves significant risks and extended recovery times. Non-surgical repair involves significant risks and recovery times. Current endoscopic closure techniques include clips, stents, and sutures. Clips can be effective for small perforations but are ineffective for large or irregular shaped defects due to their superficial degree of attachment. Stents can be useful for larger defects but have migration risks and cause patient discomfort. Fully covered self-expanding metal stents have a high migration rate, which often requires re-intervention. Sutures are technically demanding and require specialized training to provide effective results. Over-the-scope clips are designed for full-thickness closure of GI wall lesions, including perforations, fistulas, and leaks. The over-the-scope clip system can secure a larger volume of tissue and provide higher stability at a lesion site due to Nitinol material. However, the endoscope must be removed and reloaded, which can increase procedure time and introduce complexities in accessing parts of the GI tract. Therefore, there is a need for improved endoscopic systems and methods to reduce the risk of current endoscopic methods and systems while improving the treatment of iatrogenic perforations.

Type 2 Diabetes Mellitus is characterized by insulin resistance and inadequate insulin production, leading to hyperglycemia and related complications including cardiovascular disease, neuropathy, nephropathy, and retinopathy. The duodenum is a metabolic signaling center that responds to nutrients and other substances exiting the stomach. The duodenal mucosa exhibits abnormal hypertrophy and endocrine hyperplasia when a patient is suffering from Type 2 Diabetes Mellitus. Current treatment includes lifestyle modifications, pharmacologic inventions, and bariatric surgery. Many patients struggle to achieve adequate glycemic control because of poor compliance with complex medical regimens and limited efficacy of existing therapies. There is a need for a minimally invasive method for duodenal mucosal resurfacing to treat Type 2 Diabetes Mellitus.

### FIELD OF THE DISCLOSURE

The present disclosure is generally directed to endoscopic systems and devices, more specifically to endoscopic systems and devices for gastrointestinal reshaping and resurfacing via band-assisted pressure necrosis.

### BRIEF SUMMARY OF THE DISCLOSURE

The present disclosure relates to medical devices and methods for the non-surgical treatment of gastroesophageal reflux disease (GERD), obesity, Type 2 Diabetes Mellitus, and perforations through endoscopic procedures. The endoscopic procedure utilizes endoscopic barrels and a retention band for therapeutic reshaping of gastrointestinal organs. The retention band is designed to create an optimal radial force on tissue received in the retention band to ensure proper healing while also performing safe pressure necrosis.

In some embodiments, a minimally invasive endoscopic band ligation method of treating refractory GERD is disclosed. The endoscopic band ligation method places a ligation band around the LES and narrows the LES by constricting the target site. In some embodiments, band ligation is up to about 280 degrees around the LES by using an endoscopic insertion tube as a guide.

In some embodiments, a method for endoscopic band gastroplasty (EBG) and Anti Reflux Band Mucosectomy (ARBM) utilizing ligation bands to create pressure necrosis to reshape a target organ (e.g., esophagus, LES, stomach) is disclosed. The ligation band is designed to retain the pseudopolyps including the submucosa and muscle layers. The ligation band may grab a portion of the submucosa and/or the muscle layer. The ligation band is operable to capture a tissue area (e.g., cylinder) with minimum slippage and retain the tissue for a duration of time for pressure necrosis to be effective. For example, and without limitation, pressure necrosis can include controlled ischemic tissue death resulting from sustained compression by the ligation band, leading to cellular hypoxia and subsequent tissue breakdown. The greater the height of tissue above the band increases the potential for an effective mechanical lock. The width of tissue above the band increases the mechanical locking. The ligation band generates sufficient compression to assist with the capture and retention of tissue.

In some embodiments, the ligation band allows tissue to extrude through gaps in the side walls of the band. This creates a mechanical lock as the tissue expands into the space, thereby preventing the ligation band from slipping. The pressure exerted by the ligation band is from both an external surface and internal surface on the tissue that passes through the gaps of the ligation band. As a result, the pressure force is distributed across the tissue and reduces the risk of tissue damage or necrosis that typically occurs because of concentrated pressure points. By allowing the tissue to fill the gaps, the ligation band is adaptable for numerous environments and procedures. The reduced tissue slippage, improved tissue retention, and natural pressure distribution of the ligation band reduces tissue trauma, thereby improving recovery outcomes and lowering the risk of complications (e.g., inflammation or infection).

In some embodiments, a gastrointestinal reshaping endoscopic system is disclosed. The gastrointestinal reshaping endoscopic system is designed to capture and retain large tissue volumes (e.g., at least 750 mm³) via non-resection banding. The gastrointestinal reshaping endoscopic system applies pressure necrosis while capturing a muscle fiber layer. The gastrointestinal reshaping endoscopic system further reshapes the lower esophageal sphincter (e.g., ARBM) for GERD and the greater curvature of the stomach for obesity (e.g., EBG) through natural tissue remodeling.

In some embodiments, the gastrointestinal reshaping endoscopic system includes an endoscopic barrel for GERD. The GERD endoscopic barrel includes a diameter of about 12.5 mm, a depth of about 13 mm, and a volume of about 1470 mm³. In some embodiments, the gastrointestinal reshaping endoscopic system includes an endoscopic barrel for treating obesity. The obesity endoscopic barrel includes a diameter of about 15 mm, a depth of about 13 mm and a volume of about 2296 mm³.

In some embodiments, the gastrointestinal reshaping endoscopic system includes an endoscopic band and/or clip for ARBM and EBG procedures. The endoscopic band and/or clip is designed for capturing and securely holding muscular and mucosal layers, thereby providing effective pressure necrosis. The endoscopic band and/or clip further includes anti-slippage properties and a high-retention design that improves reshaping of the LES and the greater curvature of the stomach without the removal of tissue. Advantageously, this reduces the risk of complications associated with resective methods and systems.

In some embodiments, the gastrointestinal reshaping endoscopic device includes a medical device with anti-slippage properties that is desired to apply uniform pressure to a target area of tissue. The medical device is operable for gastrointestinal organ reshaping and includes an endoscopic barrel and a ligation band. The endoscopic barrel includes a volume that is sufficient to capture multiple layers of tissue. The ligation band is operable for band-assisted non-resective modification of gastrointestinal organs. The gastrointestinal reshaping endoscopic device increases the tissue capacity within the endoscopic barrel, thereby enhancing the reshaping effects without surgical resection.

In some embodiments, a method for non-resective reshaping of a gastrointestinal organ is disclosed. The method for non-resective reshaping of a gastrointestinal organ includes (1) capturing a substantial volume of tissue within an endoscopic barrel to maximize contact surface area and reshaping effect, (2) securing the suctioned tissue using a ligation band to provide stability, and (3) inducing necrosis via sustained pressure. The endoscopic barrel is operable to capture and retain tissue volumes greater than standard endoscopic barrels, thereby enhancing the effectiveness of the reshaping process.

In some embodiments, a method for treating a patient by reshaping a body lumen or passageway without tissue removal is disclosed. The method includes banding and capturing the submucosal and mucosal layers to create a fibrotic effect to reshape the LES and stomach. In some embodiments, the method further includes banding and capturing of the muscle layer into a pseudopolyp. Advantageously, the method does not include resection or removal of tissue and eliminates the risk of perforation of the muscle wall.

In some embodiments, the method includes banding an amount of tissue from a body lumen wall and/or banding tissue at a treatment site to evoke an injurious stimulus to create a tissue healing response. In some embodiments, the banded tissue is limited to tissue that occurs on a luminal side of a muscular layer of a passageway wall. For example, and without limitation, the lumen walls of body passageways include the esophagus and the stomach. These lumen walls include a mucosal layer and an underlying submucosal connective tissue layer (e.g., muscular layer). The method includes banding the mucosal layer and/or the submucosal layer without causing damage to the underlying muscle layer. In some embodiments, the underlying muscle layer is also banded.

In some embodiments, a method for treating a patient by reshaping a body lumen or passageway is disclosed. The method includes selective tissue banding, which, upon healing, results in reshaping. The methods include treatment of an esophagus to improve the resistance to gastric reflux (e.g., patient experiencing GERD). In some embodiments, the method includes modifying a stomach during a surgical procedure to treat obesity. In some embodiments, the method includes identifying a reduction of a body passageway that will reduce flow through the corresponding body passageway to improve a patient's condition. The method further includes banding tissue from an inner portion of a wall defining the identified body passageway to elicit the healing response.

In some embodiments, a method of ligating tissue using a ligation band is provided. The method of ligating tissue includes pulling tissue into an inner lumen of a barrel. The ligation band is moved along the barrel until the ligation band is positioned around targeted tissue. The ligation band grips the enclosed tissue and provides anti-slipping functionality.

In some embodiments, a ligation band is used in combination with an endoscopic band gastroplasty (EBG) barrel. The EBG barrel includes a diameter of about 15 millimeters (mm), a captured tissue depth of about 13 mm, a surface area of tissue of about 177 mm², and a captured tissue volume of about 2,296 mm³. In some embodiments, the barrel includes a 12.5 mm diameter and a tissue volume of 1,470 mm³. The barrel is designed to capture more tissue and depth on the tissue layer to increase the fibrosis effect and durability during anti-reflux band mucosectomy (ARBM) and EBG procedures. The ligation band captures and retains the muscularis propria layer for generating pressure necrosis and reshaping the lower esophageal sphincter (LES) and/or the greater curve of the stomach.

In some embodiments, an endoscopic system for gastrointestinal organ reshaping and resurfacing is disclosed. The endoscopic system includes an endoscope including at least one end barrel and at least one elastomeric ligation band. The end barrel includes a clear, plastic material and a diameter of at least 9.2 millimeters and a depth of at least 9.5 millimeters. The endoscope captures tissue corresponding to a patient. The tissue includes a mucosal layer, a submucosal layer, and at least a portion of a muscularis propria layer. The at least one elastomeric ligation band is operable to band the mucosal layer, the submucosal layer, and the portion of the muscularis propria layer. The banding of the mucosal layer, the submucosal layer, and the portion of muscularis propria layer generates a controlled pressure across the banded tissue, thereby inducing ischemia followed by pressure necrosis and subsequent fibrosis. As a result, natural healing response is generated without the need for resection, ablation, cryotherapy, argon plasma coagulation (APC), radiofrequency ablation (RFA), suturing, stapling, clipping, or other invasive modalities. In some embodiments, the at least one end barrel includes a volume of at least 750 mm³. In some embodiments, the endoscopic system treats gastroesophageal reflux disease (GERD), obesity, duodenal mucosal resurfacing (DMR) for Type 2 Diabetes Mellitus, and perforation closure. In some embodiments, the at least one end barrel includes a diameter of at least 10 millimeters and a depth of at least 11 millimeters.

In some embodiments, a method for treating gastrointestinal tissue is disclosed. The method for treating gastrointestinal tissue includes (1) identifying a body passageway for narrowing to treat gastroesophageal reflux disease (GERD) or obesity, or for resurfacing to perform duodenal mucosal resurfacing (DMR) to treat Type 2 Diabetes Mellitus or perforation closure, (2) capturing, via an endoscopic device, at least one tissue area corresponding to the identified body passageway or tissue defect, (3) deploying at least one elastomeric ligation band to the at least one tissue area, and (4) banding via the at least one elastomeric ligation band, the at least one tissue area, wherein the at least one tissue area undergoes pressure necrosis to elicit a natural healing response without resection or ablation.

In some embodiments, the method for treating gastrointestinal tissue further includes treating GERD or obesity as a result of narrowing the body passageway. In some embodiments the method for reshaping gastrointestinal tissue includes reshaping a lower esophageal sphincter (LES) and the greater curvature of a stomach. In some embodiments, a method for resurfacing gastrointestinal tissue to perform duodenal mucosal resurfacing is disclosed. The method for performing duodenal mucosal resurfacing includes removing an insulin-resistant mucosal layer and promoting regeneration without narrowing the body passageway. Typical ablation methods include mucosal layer and risk damage to deeper layers of tissue. The implementation of banding as taught herein, provides controlled, localized, tissue regeneration and lowers the risk of deep tissue damage. Tissue regeneration includes but is not limited to a natural healing response involving inflammatory response, granulation tissue formation, and epithelialization resulting in tissue remodeling and functional restoration.

In some embodiments, a method for treating perforation closure is disclosed. The method for treating perforation closure includes banding the mucosal layer, submucosal layer, and at least a portion of the muscularis propria layer to close and seal perforation and regenerate healthy tissue. In some embodiments, an endoscopic device captures at least one tissue area via suction. In some embodiments, the natural healing response improves a patient's condition. The patient's condition includes at least one of gastroesophageal reflux disease (GERD), obesity, Type 2 Diabetes Mellitus, and perforation closure.

In some embodiments, the banding process involves the inclusion of at least a portion of the muscularis propria layer within a pseudopolyp. Advantageously, this enhances fibrosis and provides durable reshaping. In some embodiments, the healing process involves fibroblast and stem cell infiltration, promoting fibrosis, new connective tissue formation and re-epithelialization leading to tissue regeneration. In some embodiments, the banding process includes elastomeric ligation bands that provide optimal tissue retention and application of pressure.

In some embodiments, an endoscopic banding system for treating GERD, obesity, duodenal mucosal resurfacing (DMR) for Type 2 Diabetes, Mellitus, and closing iatrogenic perforations utilizing elastic band ligation to treat targeted tissue areas effectively is disclosed. Advantageously, the endoscopic banding system does not use resection. The endoscopic banding system includes a plurality of ligation bands. The plurality of ligation bands are placed around a targeted tissue area to form pseudopolyps. The targeted tissue is drawn into the endoscopic barrel by suction created by a suction channel in the endoscope. The suctioned tissue is banded and, as a result, blood supply and nutrients to the pseudopolyp is restricted, which causes ischemia. Prolonged ischemia can lead to necrosis. Necrosis typically starts within twenty-four (24) hours of restricted blood flow to the banded tissue. Necrosis is characterized by the irreversible damage and death of the cells in the tissue. The necrotic tissue sloughs off and fibroblasts and stem cells infiltrate the banded tissue to promote fibrosis and connective tissue formation. Next, re-epithelization (cell remodeling) occurs and leads to healthy tissue regeneration moderated by stem cell activity. Full re-epithelialization and tissue regeneration can occur after about 21 days.

In some embodiments, a method of treating GERD by remodeling LES tissue and tightening the LES tissue to restore anti-reflux function is disclosed. In some embodiments, obesity is treated by reducing the volume of the stomach by creating restrictive rows along the greater curvature, limiting food intake. In some embodiments, duodenal mucosal resurfacing for treating Type 2 Diabetes Mellitus is performed by resurfacing the mucosal lining of the duodenum to improve insulin sensitivity and glucose metabolism. In some embodiments, iatrogenic perforations are closed. The tissue edges of a perforation are suctioned into the barrel and banded, sealing the perforation or hole in the GI tract hall, preventing infection and aiding in healing.

Advantageously, the present disclosure is an improvement over current techniques because (1) the inclusion of muscle fibers, (2) reduced risk, (3) enhanced healing, and (4) enhancing stability and durability. Without resecting, the muscle layer can be captured, thereby promoting more effective fibrosis, enhancing the reshaping process. The lack of resection reduces the risk of bleeding and perforation associated with cutting muscle fibers. A natural healing process occurs, lowering the perforation risk and improving safety.

In some embodiments, the present disclosure includes addressing perforations in the GI tract. The endoscopic barrel improves tissue capture to ensure that the ligation band encompasses deeper layers of the GI tract, including the submucosa and muscularis propria layers. The ligation band provides enhanced retention and compression to promote effective healing and minimize the risk of slippage or inadequate closure. In some embodiments, the ligation band includes a plurality of channels designed to create mechanical tissue to secure tissue. The ligation band provides compression for at least 14 days to provide tissue compression and subsequent healing. This is essential for tissue regeneration and achieving a durable closure. Advantageously, the ligation band reduces procedural costs and procedure times, lowers risks of adverse events, and increases accessibility (e.g., conscious sedation).

In some embodiments, a method of duodenal mucosal resurfacing for treating metabolic diseases without permanent implants is disclosed. The band-assisted endoscopic mucosal ligation includes pressure-induced physical resurfacing of the superficial abnormal tissue layer, promoting the regrowth of normal tissue through a stem cell-mediated healing response. An area of the duodenal mucosa is captured via an endoscopic barrel (e.g., 12 mm diameter barrel), promoting uniform resurfacing, while the minimal depth (e.g., 6 mm) reduces the risk of perforation and ensures that deeper tissue layers (e.g., muscularis propria) are not involved. Advantageously, the disclosed methods and systems reduce the risk of complications such as perforation, bleeding, and post-procedural pain because of the limited tissue captured depth. The disclosed methods and systems promote uniform tissue resurfacing and tissue regeneration. The minimally invasive nature of the duodenal mucosal resurfacing leads to shorter recovery times and improved patient comfort.

In some embodiments, a method of duodenal mucosal resurfacing is disclosed. The method of duodenal mucosal resurfacing includes preparing a patient for an endoscopic procedure. An upper GI endoscope is inserted through the patient's mouth and navigated through the pylorus, duodenal bulb, arriving at the duodenum. The endoscope includes a barrel that is placed at treatment sites along the duodenal wall and suction is applied to the treatment site tissue to capture the tissue into the barrel. At least one ligation band is deployed to capture and secure the duodenal mucosa within the endoscopic barrel. After banding, the captured tissue undergoes pressure to promote tissue resurfacing and regeneration.

### BRIEF DESCRIPTION OF THE DRAWINGS

The embodiments illustrated, described, and discussed herein are illustrative of the present disclosure. As these embodiments of the present disclosure are described with reference to illustrations, various modifications or adaptations of the methods and or specific structures described may become apparent to those skilled in the art. It will be appreciated that modifications and variations are covered by the above teachings and within the scope of the appended claims without departing from the spirit and intended scope thereof. All such modifications, adaptations, or variations that rely upon the teachings of the present disclosure, and through which these teachings have advanced the art, are considered to be within the spirit and scope of the present disclosure. Hence, these descriptions and drawings should not be considered in a limiting sense, as it is understood that the present disclosure is in no way limited to only the embodiments illustrated.

FIG. 1 illustrates banding of a pseudopolyp according to one embodiment of the present disclosure.

FIG. 2 illustrates an anatomical view of a portion of a gastrointestinal tract.

FIG. 3A illustrates a cardia banding site according to one embodiment of the present disclosure.

FIG. 3B illustrates an esophagus banding site according to one embodiment of the present disclosure.

FIG. 4 illustrates a ligation band according to one embodiment of the present disclosure.

FIG. 5 illustrates a polyp formed by a ligation band according to at least one aspect of the present disclosure.

FIG. 6A illustrates a perspective view of a barrel end on an endoscope according to at least one aspect of the present disclosure.

FIG. 6B illustrates a perspective view of a barrel end on an endoscope according to at least one aspect of the present disclosure.

FIG. 6C illustrates a side view of a barrel end on an endoscope according to at least one aspect of the present disclosure.

FIG. 6D illustrates a side view of a barrel end on an endoscope according to at least one aspect of the present disclosure.

FIG. 6E illustrates a view of the barrel end on the endoscope according to FIG. 6C along line A-A.

### DETAILED DESCRIPTION

These descriptions are presented with sufficient details to provide an understanding of one or more particular embodiments of broader inventive subject matters. These descriptions expound upon and exemplify particular features of those particular embodiments without limiting the inventive subject matters to the explicitly described embodiments and features. Considerations in view of these descriptions will likely give rise to additional and similar embodiments and features without departing from the scope of the inventive subject matters. Although the term “step” may be expressly used or implied relating to features of processes or methods, no implication is made of any particular order or sequence among such expressed or implied steps unless an order or sequence is explicitly stated.

Any dimensions expressed or implied in the drawings and these descriptions are provided for exemplary purposes. Thus, not all embodiments within the scope of the drawings and these descriptions are made according to such exemplary dimensions. The drawings are not made necessarily to scale. Thus, not all embodiments within the scope of the drawings and these descriptions are made according to the apparent scale of the drawings with regard to relative dimensions in the drawings.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a,” “an,” and “the” refer to “one or more” when used in the subject specification, including the claims. Thus, for example, reference to “a device”can include a plurality of such devices, and so forth.

Unless otherwise indicated, all numbers expressing quantities of components, conditions, and so forth used in the specification and claims are to be understood as being modified in all instances by the term “about. ” Accordingly, unless indicated to the contrary, the numerical parameters set forth in the instant specification and attached claims are approximations that can vary depending upon the desired properties sought to be obtained by the presently disclosed subject matter.

In some embodiments, the gastrointestinal reshaping endoscopic system includes an endoscopic ligator barrel and at least one ligation band. The endoscopic ligator barrel is operable to receive the at least one ligation band. In some embodiments, a pull line is operable to deploy the one or more ligation bands. The gastrointestinal reshaping endoscopic system further includes an endoscope.

In some embodiments, the gastrointestinal reshaping endoscopic system is operable for narrowing a body passageway. The narrowing of the body passageway includes (1) identifying a body passageway corresponding to a patient's medical condition, (2) determining that narrowing of a luminal diameter of the corresponding body passageway will decrease flow through the corresponding passageway, (3) determining that the decreased flow will provide relief and/or improve a patient's medical condition, (4) determining an amount of reduction to provide effective treatment, and (5) using an endoscopic device to band a portion of the luminal surface of the corresponding body passage to elicit a healing response. The method for narrowing a body passageway does not resect or remove tissue, thereby reducing risk associated with cutting and removing tissue.

In some embodiments, the gastrointestinal reshaping endoscopic system is operable to band patient tissue using an endoscope and a ligation band to modify an esophagus of a patient to reduce reflux of gastric contents into the esophagus or to modify the cardia of a patient to treat obesity. In some embodiments, the gastrointestinal reshaping endoscopic system includes conducting an endoscopic procedure that includes banding a patient's esophagus and/or cardia to reduce gastroesophageal reflux or obesity. In some embodiments, an endoscopic procedure including banding patient tissue to modify (e.g., reshape) the GEJ of a patient to treat GERD is disclosed. In some embodiments, an endoscopic procedure including banding patient tissue to modify (e.g., reshape) the cardia of a patient to treat obesity is disclosed. For example, and without limitation, in some embodiments, an endoscopic procedure for banding multiple segments of LES tissue and/or multiple rows of banded tissue for treating EBG is disclosed.

In some embodiments, a method for narrowing a body passageway is disclosed. The method includes (a) determining a first treatment area along a body passageway, (b) isolating a first portion of the mucosa, submucosa, and muscular layer using an endoscopic device, (c) banding the first portion of the mucosa, submucosa, and muscle layer, (d) moving circumferentially of the body passageway adjacent to the first banded portion, to a second portion, and (e) banding the second portion of the mucosa, submucosa, and muscle layer.

In some embodiments, the method for narrowing a body passageway bands luminal tissue to create a pressure area that promotes healing of the luminal tissue. The narrowing of the body passageway includes decreasing a diameter of the body passageway, creating folds or irregular surfaces, and/or altering the contour of a body lumen wall.

In some embodiments, the banding an amount of tissue in at least one body lumen wall to reshape a target body lumen is disclosed. For example, and without limitation, in some embodiments, at least two target tissue areas are banded. In some embodiments, the target tissue areas are separated by undisturbed tissue. In some embodiments, the target tissue areas overlap. The banding of the tissue areas results in a decrease in the diameter of a body lumen passageway. In some embodiments, the body lumen passageway diameter is reduced by at least ten percent.

For example, and without limitation, in some embodiments, the present disclosure includes a procedure for banding tissue in a first tissue area (e.g., above the LES) and a second tissue area (e.g., below the LES). The amount of banded tissue can be the same. In some embodiments, the amount of banded tissue varies for each tissue area. For further example, and without limitation, in some embodiments, a first banded tissue area is between about 1% and about 50% greater than a second banded tissue area.

When reshaping the GEJ to treat GERD, tissue can be removed near, above, and/or below the GEJ to induce a healing response. When treating obesity, one or more regions of the stomach (e.g., gastric cardia) can be banded.

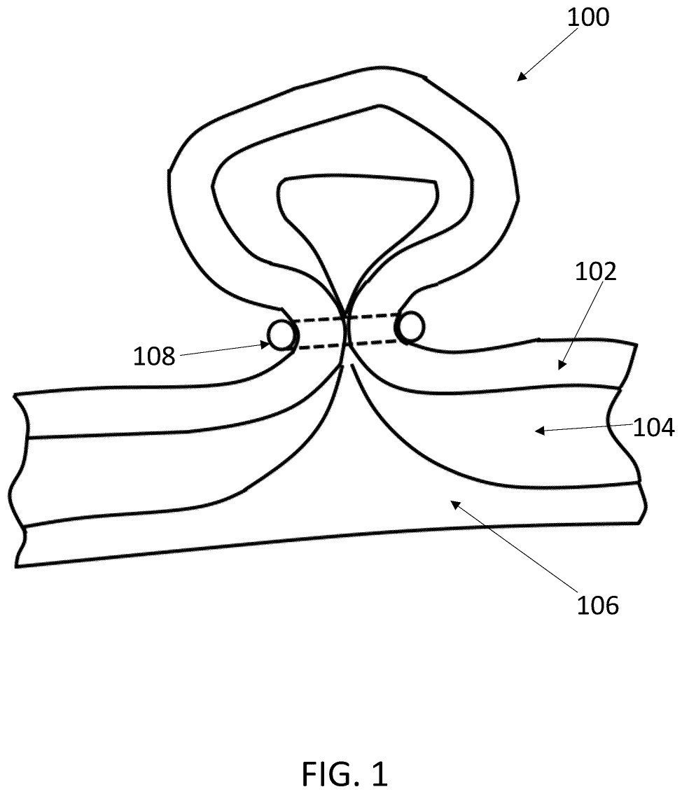

FIG. 1 illustrates a representation of a pseudopolyp 100 including a mucosa layer 102, a submucosa layer 104, and a muscle layer 106. The pseudopolyp 100 is banded by a ligation band 108. The ligation band 108 can be used in combination with an endoscope to capture and band tissue. In some embodiments, suction is provided and draws the mucosa layer 102, the submucosa layer 104, and the muscle layer 106 to the ligation band 108. The ligation band is deployed and bands the captured tissue. In some embodiments, a plurality of ligation bands is used. The ligation band is capable of capturing and retaining muscularis propria for pressure necrosis for the purpose of re-shaping LES and the greater curve of the stomach.

In some embodiments, endoscopic systems and methods for treating the gastroesophageal junction (GEJ), lower esophageal sphincter (LES), and the squamocolumnar junction (SCJ) is disclosed. In some embodiments, the endoscopic systems and methods include a plurality of banded tissue areas below the GEJ. For example, and without limitation, the number of banded tissue areas includes at least two. By banding the tissue (e.g., cardia) below the GEJ, a patient will have a smaller passageway for food intake and should experience satiety at a faster rate compared to prior to the banding procedure.

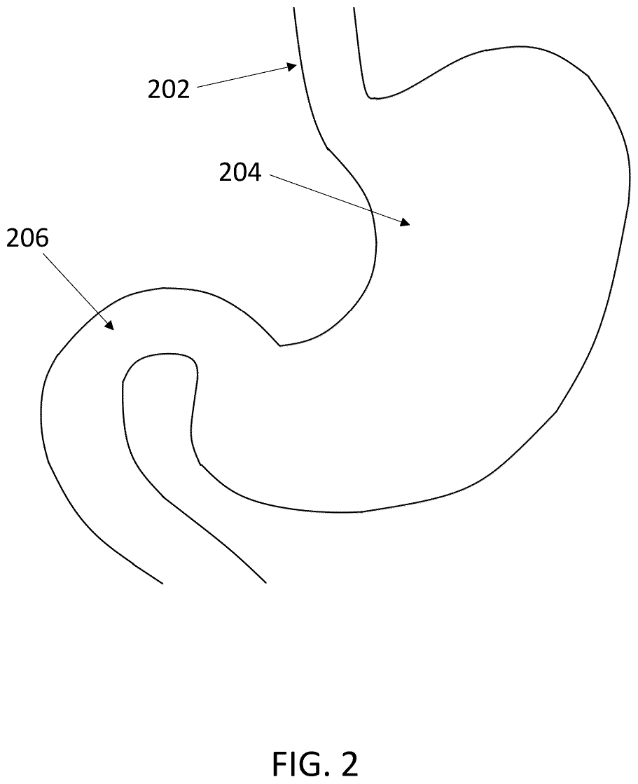

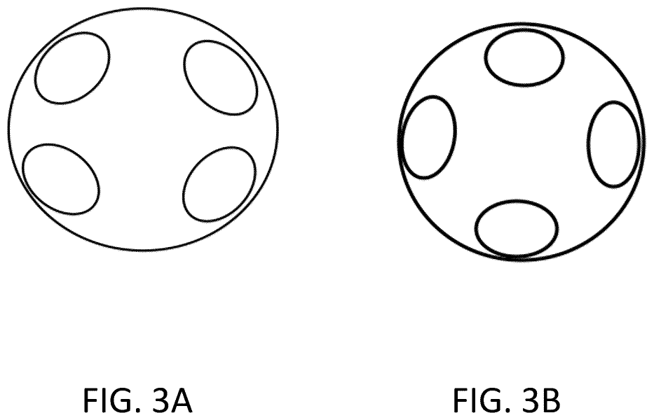

FIG. 2 illustrates an esophagus 202, a stomach 204, and a duodenum 206 of a gastrointestinal tract. For example, and without limitation, in some embodiments, a procedure and system to reshape and narrow a body passageway positioned between the esophagus and the stomach is disclosed. Advantageously, the reshaping and narrowing of the body passageway between the esophagus and the stomach can provide a barrier against reflux of gastric contents. In some embodiments, the gastric cardia below the GEJ is banded to reduce food intake to improve weight loss and treat obesity. FIG. 3A illustrates banding sites for a cardia near the squamocolumnar junction. FIG. 3B illustrates banding sites for an esophagus near the squamocolumnar junction.

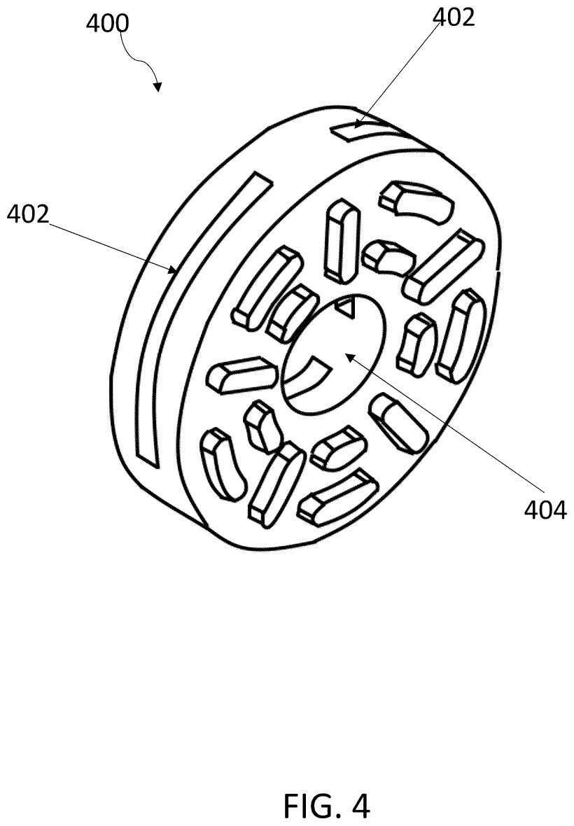

In some embodiments, as shown in FIG. 4, a ligation band 400 is disclosed. The ligation band 400 includes a plurality of channels 402 and at least one hole 404. The plurality of channels 402 includes at least three channels. In some embodiments, the plurality of channels includes a tapered shape, a curved shape, a rectangular shape, and other polygonal shapes or surface modifications increasing friction. The plurality of channels 402 is connected to the at least one hole 404. For example, and without limitation, each channel of the plurality of channels is connected to the at least one hole at about halfway between the top and bottom of the at least one hole. Each channel of the plurality of channels is designed to receive tissue that passes through the at least one hole 404. For example, and without limitation, the plurality of channels is operable to mechanically lock tissue received from the at least one hole. For further example, tissue can squeeze through the plurality of channels and press against all walls of a channel. The at least one hole 404 runs (e.g., from top to bottom) through a central portion of the ligation band 400. Advantageously, when performing ligation, the at least one hole 404 passes compressed tissue through the ligation band 400. The ligation band 400 is operable to generate a uniform force of pressure to the captured tissue to improve pressure necrosis and a corresponding healing response.

In some embodiments, the plurality of channels includes at least one gripping feature. The at least one gripping feature is designed to frictionally hold tissue. The at least one gripping feature can be a cylindrical shape, a rectangular shape, a conical shape, and other similar polygonal shapes. In some embodiments, the ligation band includes a rough surface for frictionally holding tissue. In some other embodiments, the ligation band includes a rough surface and discreet gripping features.

In some embodiments, each channel is equally spaced apart. Alternatively, the plurality of channels is not equally spaced part. In some embodiments, the ligation band further includes a plurality of side channels. The plurality of side channels is connected to a central hole of the ligation band and enables the tissue to extend to the side of the ligation band. For further example, and without limitation, each side channel of the plurality of side channels is vertically adjacent to each other and connected to the same side of the hole. In some embodiments, vertically adjacent side channels are relative to the height of the ligation band.

In some embodiments, the ligation band is a unitary piece. Alternatively, the ligation band is at least two separate pieces that are removably attached to each other. For example, and without limitation, a bottom component of the ligation band includes a plurality of holes for receiving an attachment component (e.g., pin) of a top component of the ligation band Alternatively, or additionally, a top component and a bottom component of a ligation band are adhesively attachable. In some embodiments, the plurality of protrusions is positioned on both a top and bottom of the ligation band.

In some embodiments, a ligation band designed for minimizing slippage and retaining tissue for pressure necrosis is disclosed. The ligation band captures tissue and retains the tissue in place for a duration of time. For example, and without limitation, the plurality of channels is operable to mechanically lock tissue received from the at least one hole. For further example, tissue can squeeze through the plurality of channels and presses against all walls of a channel. The depth and width of tissue above the band and inside tissue receiving channels increases the potential for a more effective mechanical lock. The ligation band is designed to generate an adequate compression force to assist with the capture and retention of tissue. The ligation band includes surface elements to assist with anti-slippage. In some embodiments, the ligation band further includes a plurality of gripping features designed to improve the capture and retention of tissue by the ligation band. In some embodiments, a method including endoscopic placement of ligation bands for the approximation of soft tissue for primary gastric restrictive procedures is disclosed. A minimally invasive, accessible endoscopic band ligation approach for the treatment of morbid obesity.

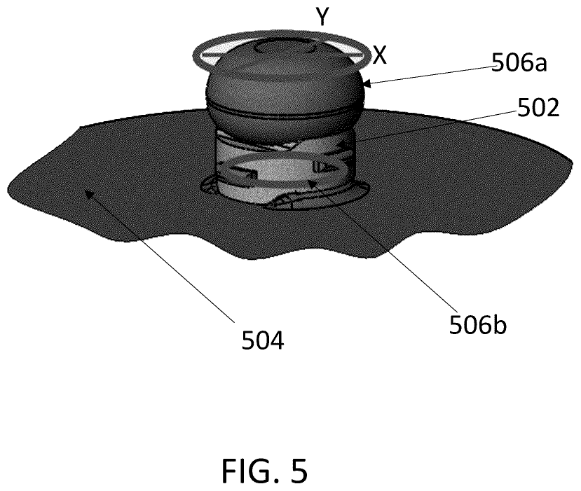

FIG. 5 illustrates a pseudopolyp formed by a ligation band according to at least one aspect of the present disclosure. Once the ligation band 502 bands arounds the tissue 504, a pseudopolyp head 506a and a pseudopolyp neck 506b is formed. The ligation band 502 is configured to generate a radial force on the tissue 504. The radial force can create a desired pseudopolyp head-to-neck ratio. For example, and without limitation, the desired pseudopolyp head-to-neck ratio is at least 1.25. This pseudopolyp head-to-neck ratio creates a mechanical tissue lock above the ligation and provides a minimum threshold for tissue retention. The radial force may be at least 3 Newtons.

In at least one aspect of the present disclosure, a diameter of the pseudopolyp head 506a is greater than the diameter of the pseudopolyp neck 506b. The diameter of the pseudopolyp head 506a can be determined as an average of measurement in an x and y direction. For example, and without limitation, the ligation band may be designed to generate a pseudopolyp neck of at least 4 millimeters. In another example, the ligation band may be designed to generate a pseudopolyp neck between about 4 millimeters and 8 millimeters or about 6 millimeters and about 8 millimeters. The ligation band is configured to apply a desired radial force (e.g., 3 Newtons) to a pseudopolyp (e.g., pseudopolyp neck of at least 6 millimeters) for at least 48 hours.

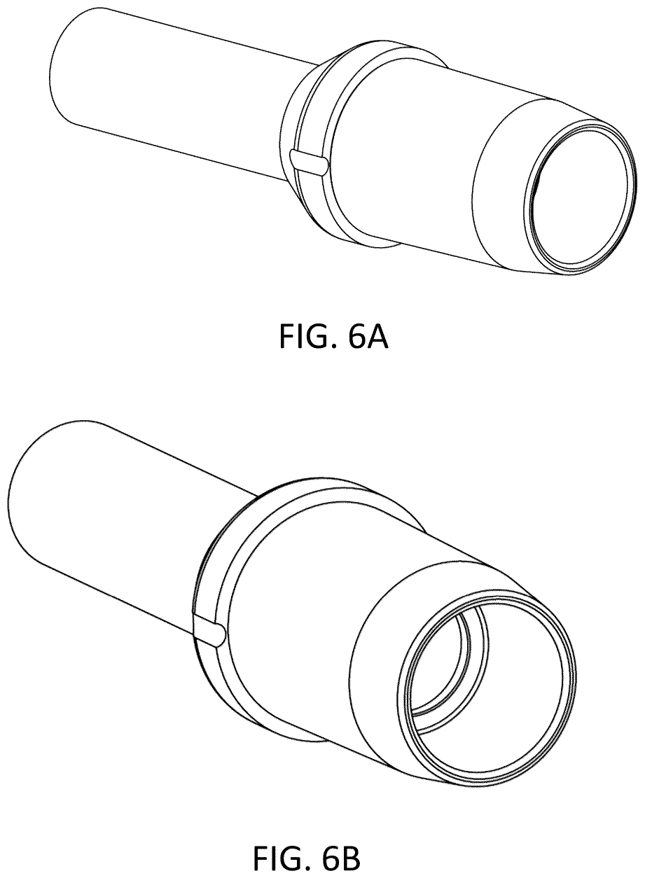

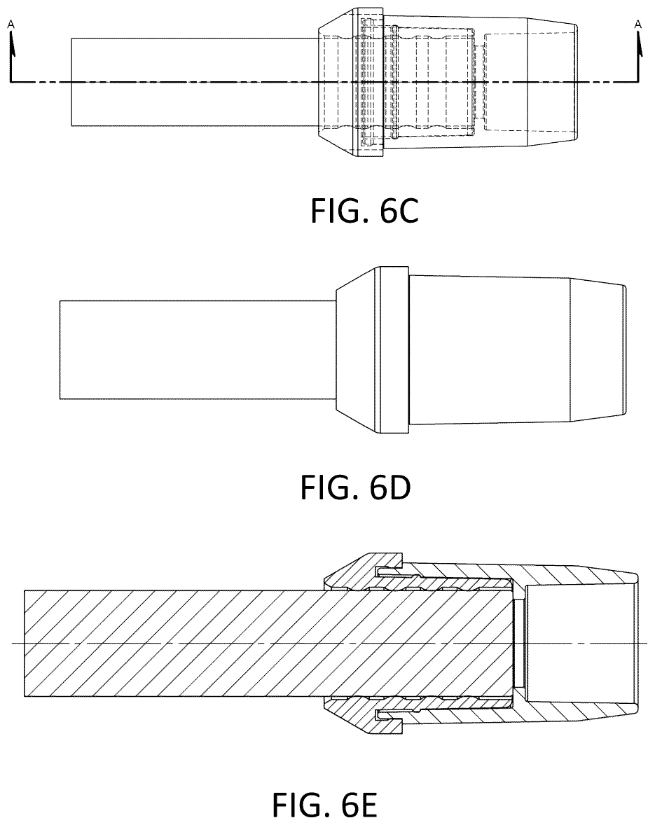

FIG. 6A illustrates a perspective view of a barrel end on an endoscope according to at least one aspect of the present disclosure. FIG. 6B illustrates a perspective view of a barrel end on an endoscope according to at least one aspect of the present disclosure. FIG. 6C illustrates a side view of a barrel end on an endoscope according to at least one aspect of the present disclosure. FIG. 6D illustrates a side view of a barrel end on an endoscope according to at least one aspect of the present disclosure. FIG. 6E illustrates a view of the barrel end on the endoscope according to FIG. 6C along line A-A.

The corresponding structures, materials, acts, and equivalents of all means or step plus function elements in the claims below are intended to include any structure, material, or act for performing the function in combination with other claimed elements as specifically claimed.

Particular embodiments and features have been described with reference to the drawings. It is to be understood that these descriptions are not limited to any single embodiment or any particular set of features, and that similar embodiments and features may arise or modifications and additions may be made without departing from the scope of these descriptions and the spirit of the appended claims.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

What is claimed is:

1. An endoscopic system for gastrointestinal reshaping, the endoscopic system comprising:
an endoscope including at least one end barrel; and
at least one ligation band;
wherein the endoscope is operable to capture a tissue area corresponding to a patient, wherein the captured tissue area includes at least one of a mucosal layer, a submucosal layer, or a portion of a muscular layer; and
wherein the at least one ligation band is operable to band the captured tissue area; and
wherein, once the captured tissue area is banded, the at least one ligation band generates pressure across the banded tissue, wherein the banded tissue undergoes pressure necrosis to elicit a healing response.

2. The gastrointestinal reshaping endoscopic system of claim 1, wherein the at least one end barrel includes a volume of at least 750 mm³.

3. The gastrointestinal reshaping endoscopic system of claim 1 further configured to treat gastroesophageal reflux disease (GERD), type 2 diabetes mellitus (T2DM), and/or obesity.

4. The gastrointestinal reshaping endoscopic system of claim 1, wherein the at least one end barrel includes a diameter of at least 12 mm and a depth of at least 12 mm.

5. The gastrointestinal reshaping endoscopic system of claim 1, wherein the at least one end barrel includes a transparent plastic material, wherein the at least one end barrel further includes a diameter of at least 9.2 millimeters and a depth of at least 9.5 millimeters.

6. The gastrointestinal reshaping endoscopic system of claim 5, wherein the at least one end barrel includes a volume of at least 750 mm³.

7. The gastrointestinal reshaping endoscopic system of claim 5, wherein the healing response improves a condition of a patient, wherein the patient condition includes at least one of gastroesophageal reflux disease (GERD), obesity, Type 2 Diabetes Mellitus, or a perforation closure.

8. The gastrointestinal reshaping endoscopic system of claim 1, wherein, when the captured tissue area is banded, a pseudopolyp is formed, wherein the pseudopolyp includes an upper portion above the at least one ligation band, wherein the pseudopolyp includes a portion inside the at least one ligation band, wherein a diameter of the upper portion is greater than a diameter of the inner portion of the pseudopolyp.

9. A method for reshaping gastrointestinal tissue comprising:
identifying a body passageway for narrowing, wherein narrowing the body passageway will improve a patient's condition;
capturing, via an endoscopic device, at least one tissue area corresponding to the identified body passageway;
deploying at least one ligation band to the at least one tissue area; and
banding, via the at least one ligation band, the at least one tissue area, wherein the at least one tissue area undergoes pressure necrosis to elicit a healing response.

10. The method of reshaping gastrointestinal tissue of claim 9, wherein the method for reshaping gastrointestinal tissue does not include resecting the at least one tissue area.

11. The method of reshaping gastrointestinal tissue of claim 10, further comprising reshaping a lower esophageal sphincter (LES) and the greater curvature of a stomach.

12. The method of reshaping gastrointestinal tissue of claim 9, wherein the endoscopic device captures the at least one tissue area via suction.

13. The method of reshaping gastrointestinal tissue of claim 9, wherein the healing response improves a patient's condition, wherein the patient's condition includes gastroesophageal reflux disease (GERD) or obesity.

14. The method of reshaping gastrointestinal tissue of claim 9, wherein the at least one tissue area includes at least one of a mucosal layer, a submucosal layer, or a portion of a muscular layer.

15. A method of treating gastrointestinal tissue comprising:
identifying a body passageway to treat at least one patient condition, wherein the at least one patient condition includes at least one of gastroesophageal reflux disease (GERD), obesity, Type 2 Diabetes Mellitus, or perforation closure;
capturing, via an endoscopic device, at least one tissue area corresponding to the identified body passageway;
deploying at least one elastomeric ligation band to the at least one tissue area; and
banding, via the at least one elastomeric ligation band, the at least one tissue area, wherein, as a result of the banding, the at least one tissue area undergoes pressure necrosis to elicit a natural healing response.

16. The method of treating gastrointestinal tissue of claim 15 further comprising narrowing the identified body passageway to treat GERD and/or obesity.

17. The method of treating gastrointestinal tissue of claim 16 further comprising reshaping a lower esophageal sphincter (LES) and the greater curvature of a stomach.

18. The method of treating gastrointestinal tissue of claim 17, wherein an insulin-resistant mucosal layer is removed, wherein the identified body passageway is narrowed, wherein, as a result of the narrowed identified body passageway, the at least one tissue area undergoes tissue regeneration.

19. The method of treating gastrointestinal tissue of claim 15 further comprising banding a mucosal layer, a submucosal layer, and at least a portion of a muscularis propria layer of the at least one tissue area.

20. The method of treating gastrointestinal tissue of claim 15, wherein the endoscopic device captures the at least one tissue area via suction.
